(19) United States
(12) Patent Application Publication
(10) Pub. No.: US 2020/0397283 A1
(43) Pub. Date: Dec. 24, 2020

(54) A METHOD TO QUANTIFY THE CORNEAL PARAMETERS TO IMPROVE BIOMECHANICAL MODELING

(71) Applicant: Narayana Nethralaya Foundation, Bangalore (IN)

(72) Inventors: Abhijit Sinha Roy, Bangalore (IN); Rohit Shetty, Bangalore (IN); Mathew Francis, Bangalore (IN); Rachana Chandapura, Bangalore (IN)

(73) Assignee: Narayana Nethralaya Foundation, Bangalore (IN)

(21) Appl. No.: 16/975,005

(22) PCT Filed: Jan. 18, 2019
(86) PCT No.: PCT/IB2019/050661

§ 371 Date: Aug. 21, 2020

(30) Foreign Application Priority Data
Feb. 28, 2018 (IN) 201841007518

(51) Int. Cl. A61B 3/107; A61B 34/10; G16H 30/40; G16H 50/20; G16H 50/50

(52) U.S. Cl. CPC A61B 3/107; A61B 34/10; A61B 2034/105; G16H 50/20; G16H 50/50; G16H 30/40

(57) ABSTRACT

The invention relates to a method to quantify human corneal tissue parameters to improve biomechanical modelling of refractive and therapeutic procedures to reduce or eliminate refractive errors and unwanted wavefront aberrations. The analysis of the preoperative biomechanical properties of cornea assists in predicting the postoperative biomechanical response of cornea. The computational models of extreme properties of the corneal tissue are conducted and the 3-D finite element models are populated with tomographic information of the cornea and artificial intelligence is trained to consider the pre-operative characteristics of the corneal tissue. The post-operative refractive procedures are simulated using the said finite element models. Finally, biomechanical deformations are prospectively estimated and method builds a deep learning approach to link clinical features to changes in biochemical outcomes. The method predicts the post-operative biomechanics of the corneal tissue and derives long term postoperative biomechanical response of cornea.

### DESCRIPTION OF THE INVENTION

### Technical Field of the Invention

The present invention relates to a method to quantify the human corneal tissue parameters to improve the biomechanical modelling of refractive and therapeutic procedures meant to reduce or eliminate refractive errors and unwanted wavefront aberrations.

### BACKGROUND OF THE INVENTION

Cornea is the outermost layer of the eye, which is transparent, dome-shaped surface that covers the front part of the eye. Cornea receives nourishment from the tears and the aqueous humor that fills the chamber behind it due to absence of highly organized group of cells and blood vessels.

Cornea, the transparent anterior part of the eye covers the iris, pupil and an anterior chamber. Cornea together with the lens refracts light accounting for approximately two-thirds of the eye's total optical power.

Cornea has unmyelinated nerve endings, which are sensitive to stimuli, temperature and chemicals and causes an involuntary reflex to close the eyelid. The refractive power of the cornea in humans is approximately 43 dioptres. In humans, the cornea has a diameter of 11.5 mm and a thickness of 500-600 μm in the center and 600-800 μm at the periphery. The characteristics such as transparency, avascularity, the presence of immature resident immune cells and immunologic privilege makes the cornea a very special tissue.

Cornea comprises corneal epithelium, Bowman's layer, corneal stroma, Descemet's membrane and corneal endothelium. The anterior surface of the cornea is elliptical whereas posterior surface is spherical with average diameter 11.5 mm. The horizontal diameter of the anterior surface is 11.7 mm and vertical diameter is 11 mm. The radius of curvature of cornea is 8 mm. The central corneal thickness is 0.52 mm, whereas periphery is 0.67 mm thick. Central corneal thickness directly influences the measurement of intraocular pressure. The central 5 mm of the cornea forms the most powerful refracting surface of the eye.

During any injury in corneal epithelium, the basal cells are moved to the damaged area and subsequent cellular proliferation from basal layers fills the defect. Bowman's membrane does not regenerate. The wound in Bowman's membrane is replaced by stroma like fibrous tissue or epithelium. In response to trauma to corneal stroma, there is increase in number of keratinocytes and the ground substances and collagen fibrils secreted by the keratinocytes. However, the stromal tissue thus produced is different and the diameter of the newly formed stromal collagen is larger than the normal stromal collagen. Further, the newly formed collagen fibrils are not well organized and lack the normal tensile strength of the collagen fibrils.

Refractive surgery and corneal transplant are the two surgeries designed to deliver therapeutic treatment to the patient. Refractive surgery improves the refractive state of the eye and decreases or eliminates the dependency on glasses or contact lenses. This also includes various methods of surgical remodeling of the cornea such as topography guided treatments or cataract surgery. Corneal transplant is a surgical procedure where a damaged or diseased cornea is replaced by donor corneal tissue. These surgeries are conducted with manual technique or with the use of laser. The surgery results in alteration of the biomechanical properties of the cornea resulting in change in tomography of the cornea as well. The knowledge of the preoperative biomechanical properties of the cornea assists in predicting the postoperative biomechanical response of the cornea.

The anterior stroma of the cornea has an interweaving network of collagen fibers around the collagen lamellae. This interweaving progressively becomes less through the depth of the stroma. These structural features are responsible for the greater tensile strength of the anterior stroma relative to the posterior stroma.

The US patent application “US20080086048A1” titled “Method for measuring biomechanical properties in an eye” discloses a system and method for characterizing the biomechanical properties of tissue within an eye. A perturbation component introduces a stress to the eye tissue. An imaging component is operative to obtain an image of the eye tissue. A first image of the tissue is obtained prior to the introduction of the stress and a second image of the tissue is obtained after the introduction of the stress. An image analysis component compares the first image and the second image as to determine at least one biomechanical property of the tissue. However, the method is incomplete to predict the post-operative biomechanical changes in the corneal tissue.

The US patent application “US20080262610A1” titled “Biomechanical design of intracorneal inlays” discloses intracorneal inlays for correcting vision impairments by altering the shape of the anterior corneal surface. The physical design of the inlay to induce the desired change of the anterior corneal surface, which includes consideration of the biomechanical response of the corneal tissue to the physical shape of the inlay. This biomechanical response differs depending on the thickness, diameter and profile of the inlay. The inlays having diameters smaller than the pupil are provided for correcting presbyopia. To provide the near vision, an inlay is implanted centrally in the cornea to induce an “effect” zone on the anterior corneal surface, within which diopter power is increased whereas the distance vision is provided by a region of the cornea peripheral to the “effect” zone. The intracorneal inlay design induces a multi-diopter power multifocal effect for the correction of correction of simple refractive error. However, the method is incomplete to predict the post-operative biomechanical changes in the corneal tissue.

The US patent application “U.S. Pat. No. 7,130,835B2” titled “System and method for predictive ophthalmic correction” discloses a system and method for providing a predictive outcome in the form of a predictive best instruction for a therapeutic ophthalmic correction of a patient's vision defects. The predictive best instruction is derived from prospective therapeutic-outcome-influencing, new information that is analyzed in conjunction with optimized, historical therapeutic-outcome information. The instruction is preferably an optimized, custom, photoablative algorithm for driving a photoablative, laser. However, the method is incomplete to predict the post-operative biomechanical changes in the corneal tissue.

The U.S. Pat. No. 5,891,131A titled “Method and apparatus for automated simulation and design of corneal refractive procedures” discloses a technique for automated design of a corneal surgical procedure, which includes topographical measurements of a patient's eye to obtain corneal surface topography. Generally, the conventional techniques are used to obtain the thickness of the cornea and the intraocular pressure. The topographical information is interpolated and extrapolated to fit the nodes of a finite element analysis model of the eye, which is then analyzed to predict the initial state of stress of the eye and obtain pre-operative curvatures of the cornea. Incision data constituting the “initial” surgical plan is incorporated into the finite element analysis model. A new analysis is performed to simulate resulting deformations, stress and curvatures of the eye. They are compared to the original values thereof and to the vision objective. The surgical plan is modified and the resulting new incision data is entered into the model and the analysis is repeated. The procedure is repeated until the vision objectives are met. However, the method fails to predict the post-operative biomechanical changes in the corneal tissue with patient variability.

There are methods available for the quantitative analysis of cornea. However, the available methods disclose only the processes to analyse the quantitative changes. Hence, there is a need for a method to quantify the human corneal tissue parameters to improve the biomechanical modelling of refractive and therapeutic procedures meant to reduce or eliminate refractive errors and unwanted wavefront aberrations.

### SUMMARY OF THE INVENTION

The invention relates to a method to quantify the human corneal tissue parameters to improve the biomechanical modeling. The refractive surgery and corneal transplant are designed to deliver therapeutic treatment to the patient.

The knowledge and analysis of the preoperative biomechanical properties of the cornea assist in predicting the postoperative biomechanical response of the cornea.

The method of quantification involves the characterization the tomographic information of the cornea such as curvature, corneal thickness, epithelium thickness. The database of the preoperative demographics and surgical parameters are also considered. The computational models of extreme properties of the corneal tissue are conducted and the 3-D finite element models are populated with tomographic information of the cornea. Then the artificial intelligence is trained to consider the pre-operative characteristics of the corneal tissue. The post-operative refractive procedures are simulated using the said 3-D finite element models. Finally, biomechanical deformations or shape of cornea are prospectively estimated, which also includes severance of collagen fibers and alteration of hydration. The method builds a deep learning approach to link the clinical features to changes in biomechanical outcomes.

The method helps in prediction of the post-operative biomechanics of the corneal tissue and derives a long term postoperative biomechanical response of the cornea.

### BRIEF DESCRIPTION OF THE DRAWINGS

The foregoing and other features of embodiments will become more apparent from the following detailed description of embodiments when read in conjunction with the accompanying drawings.

FIG. 1 illustrates the flowchart for a method for quantitative analysis of tomographic characteristics of the cornea.

### DETAILED DESCRIPTION OF THE INVENTION

In order to make the matter of the invention clear and concise, the following definitions are provided for specific terms used in the following description.

The term “Corneal Transplantation” refers to a surgical procedure where a damaged or diseased cornea is replaced by donated corneal tissue.

The term “Biomechanical modeling” refers to the modeling by application of mechanical laws to living structures.

The present invention relates to a method to quantify the human corneal tissue parameters to improve the biomechanical modeling.

The refractive surgery and corneal transplant are designed to deliver therapeutic treatment to the patient. These surgeries are conducted with manual technique or sometimes with the use of laser technology. Mostly, the surgery results in change in biomechanical properties of the cornea resulting in change in tomography of the cornea.

The knowledge and analysis of the preoperative biomechanical properties of the cornea assist in predicting the postoperative biomechanical response of the cornea. Thus reducing the risk of complications postoperatively.

The invention discloses the method for quantitative analysis of tomographic characteristics of the cornea preoperatively. Then cornea is populated with 3-D finite element model of surgeries and these models solve to derive the postoperative biomechanical response of the cornea.

The parameters such as patient demographics, corneal tomography, corneal mechanics, age, gender, systemic conditions, genetic profile and planned treatment are characterized and considered pre-operatively.

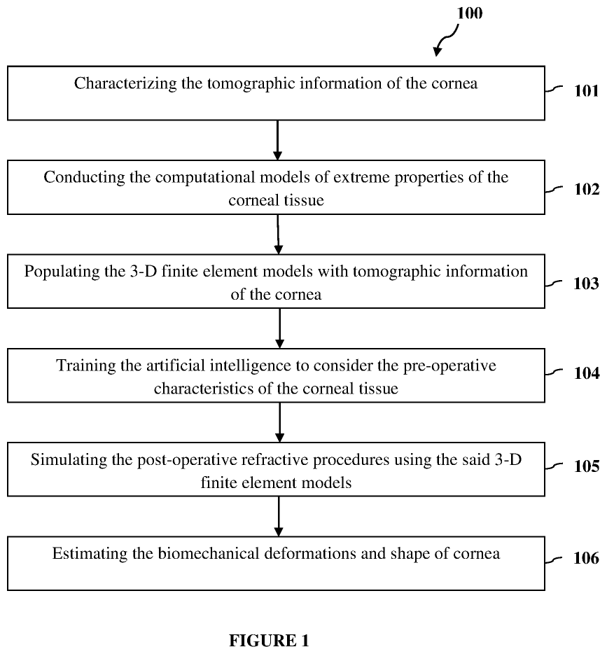

FIG. 1 illustrates the flowchart for a method for quantitative analysis of tomographic characteristics of the cornea. The method (100) of quantification initiates with step (101) of characterizing the tomographic information of the cornea. The tomographic features such as curvature, corneal thickness, epithelium thickness are characterized. The database of the preoperative demographics and surgical parameters are considered. At step (102), computational models of extreme properties of the corneal tissue are conducted. At step (103), the 3-D finite element models are populated with tomographic information of the cornea. The models include the expected changes in the structure of the cornea. At step (104), the artificial intelligence is trained to consider the pre-operative characteristics of the corneal tissue. At step (105), the post-operative refractive procedures are simulated using the said 3-D finite element models. At step (106), biomechanical deformations or shape of cornea are prospectively estimated. The estimation also includes severance of collagen fibers and alteration of hydration.

The method builds a deep learning approach to link the clinical features to changes in biomechanical outcomes. The finite element models predict the post-operative biomechanics of the corneal tissue.

The method helps in prediction of the post-operative biomechanics of the corneal tissue. The method derives the long term postoperative biomechanical response of the cornea. However, patient variability in wound healing rates is expected and pure biomechanical modeling of acute response after surgery would not be sufficient.

The method include patient data from past surgical procedures such as tomographic information including curvature, corneal thickness, epithelium thickness both pre and postoperative, which is linked through a deep learning approach to modify the predictions of the finite element modeling outcomes. This improves the accuracy of the predictions as deep learning helps in reducing the effect of variability in patient wound healing on prediction of biomechanical properties.

We claim:

1. A method for quantification of human corneal tissue parameters to improve the biomechanical modeling, the method (100) comprises the steps of:
a. characterizing the tomographic information of the cornea (101);
b. conducting the computational models of extreme properties of the corneal tissue (102);
c. populating the 3-D finite element models with tomographic information of the cornea (103);
d. training the artificial intelligence to consider the pre-operative characteristics of the corneal tissue (104);
e. simulating the post-operative refractive procedures using the said 3-D finite element models (105); and
f. estimating the biomechanical deformations and shape of cornea.

2. The method as claimed in claim 1, wherein tomographic information of the cornea characterized are curvature, corneal thickness and epithelium thickness.

3. The method as claimed in claim 1, wherein a database of the preoperative demographics and surgical parameters are considered for tomographic information.

4. The method as claimed in claim 1, wherein the model predicts the post-operative severance of collagen fibers and alteration of hydration in cornea.

5. The method as claimed in claim 1, wherein the tomographic information is linked to a deep learning approach to modify the predictions of the finite element modeling outcomes.
